(19) United States
(12) Patent Application Publication
(10) Pub. No.: US 2025/0131250 A1
(43) Pub. Date: Apr. 24, 2025

(54) SYSTEM AND METHOD FOR MEDICAL IMAGE TRANSLATION

(71) Applicants: VOLPARA HEALTH TECHNOLOGIES LIMITED, Wellington (NZ); VOLPARA SOLUTIONS EUROPE LIMITED, Stockport (GB)

(72) Inventors: Kaier WANG, Lower Hutt (NZ); Ralph HIGHNAM, Wellington (NZ)

(21) Appl. No.: 18/682,408

(22) PCT Filed: Aug. 10, 2022
(86) PCT No.: PCT/IB2022/057460

§ 371 Date: Feb. 8, 2024

(30) Foreign Application Priority Data
Aug. 10, 2021 (GB) 2111497.0

(51) Int. Cl. G06N 3/0475; G06T 7/00

(52) U.S. Cl. CPC G06N 3/0475; G06T 7/0012; G06T 2207/30068

(57) ABSTRACT

A system and method, relates to the field of medical imaging and image translation. It relates, in particular, to means to translate a for-processing image to a for-presentation image that is manufacturer and modality agnostic. It is a system and method for learning a translation mapping between for-processing and for-presentation image pairs via a generative adversarial network (GAN) based deep learning system. The Generative Adversarial Network (GAN) comprises a first neural network as a generator and a second neural network as a discriminator configured to train one another to learn a translation mapping between sets of paired for-processing and for-presentation images.

### FIELD OF THE INVENTION

Field of the invention: The present invention relates to the field of medical imaging and image translation. It relates, in particular, to means to translate a for-processing image to a for-presentation image that is manufacturer and modality agnostic.

### BACKGROUND

The present invention provides means for the translation of medical images (for example, images of the prostate, lung and breast) from ‘for-processing’ (also referred to as ‘raw’) format to ‘for-presentation’ (also referred to as ‘processed’) format that is manufacturer and modality agnostic via generative adversarial network (GAN) based deep learning system.

In radiographic imaging, a detector generates for-processing images in which the grayscale is proportional to the x-ray attenuation through the scanned body part and the internal organs or tissues. These data are then digitally manipulated to enhance some features, such as contrast and resolution, to yield for-presentation images that are optimised for visual lesion detection by radiologist.

However, radiography equipment manufacturers do not disclose their for-processing to for-presentation image conversion details. Hence, retrospective image review is not possible for most historical images (i.e. images stored only in the for-processing format due to cost and storage constraints).

Moreover, as illustrated by Gastounioti et al (′Breast parenchymal patterns in processed versus raw digital mammograms: A large population study toward assessing differences in quantitative measures across image representations. Medical Physics 2016 November; 43(11):5862. doi: 10.1118/1.4963810′), the texture characterization of the breast parenchyma varies substantially across vendor-specific for-presentation images.

Image translation refers to tasks in which an image in a source domain (for example, the domain of gray-scale images), is translated into a corresponding image in a target domain (for example, the domain of colour images), where one visual representation of a given input is mapped to another representation.

Developments in the field of image translation have been largely driven by the use of deep learning techniques and the application of artificial neural networks. Among such networks, convolutional neural networks (CNNs) have been successfully applied to medical images and tasks to distinguish between different classes or categories of images, for example, to the detection, segmentation, and quantification of pathologic conditions.

Artificial intelligence (AI) based applications also include the use of generative models. These are models that can be used to synthesize new data. The most widely used generative models are generative adversarial networks (GANs).

A GAN is an AI technique where two artificial neural networks are jointly optimized but with opposing goals. One neural network, the generator, aims to synthesize images that cannot be distinguished from real images. The second neural network, the discriminator, aims to distinguish these synthetic images from real images. The two models are trained together in an adversarial, zero-sum game, until the discriminator model is ‘fooled’ above a requisite occurrence, meaning the generator model is generating plausible examples. These deep learning models allow, among other applications, the synthesis of new images, acceleration of image acquisitions, reduction of imaging artifacts, efficient and accurate conversion between medical images acquired with different modalities, and identification of abnormalities depicted on images.

As with other deep learning models, GAN development and use entails: a training stage in which a training dataset is used to optimise the parameters of the model; and a testing stage, in which the trained model is validated and eventually deployed. In a GAN system, the first neural network generator and the second neural network discriminator are trained simultaneously to maximise their performance: the generator is trained to generate data that fail the discriminator; and the discriminator is trained to distinguish between real and generated data.

To optimise the performance of the generator, the GAN strives to maximize the loss of the discriminator given generated data. To optimize the performance of the discriminator, the GAN strives to minimise the loss of the discriminator given both real and generated data.

The discriminator may comprise separate paths which share the same network layers where each layer computes a feature map which may be described as the image information where the layer has the most attention (J. Yosinski, et al. (‘Understanding Neural Networks Through Deep Visualization’, ICML Deep Learning Workshop 2015)). Feature maps from the lower layers are found to highlight simple features such as object edges, corners. There is an increase in complexity and variation on higher layers, comprised of simpler components from lower layers.

In radiologic applications, GANs are used to synthesize images conditioned on other images. The discriminator determines for pairs of images whether they form a realistic combination. Thus it is possible to use GANs for image-to-image translation problems such as correction of motion artefacts, image denoising, and modality translation (e.g. PET to CT).

GANs also allow the synthesis of completely new images, for example, to enlarge datasets, where the synthesized data are used to enlarge the training dataset for a deep learning-based method and thus improve its performance.

GANs have also been used to address limitations of image acquisition that would otherwise necessitate a hardware innovation such as detector resolution or motion tracking. For example, a GAN could be trained for image super-resolution perhaps via increasing image matrix sizes above those originally acquired: the input image of the generator network would be a low-resolution image, and the output image of that network would be a high-resolution image.

GANs allow to some extent the synthesis of image modalities which helps to reduce time, radiation exposure and cost. For example, a generator CNN can be trained to transform an image of one modality (the source domain) into an image of another modality (the target domain). Such a transformation is typically nonlinear, and a discriminator could be used to encourage characteristics of the target domain on the output image.

Given paired images in different domains, it is possible to learn their nonlinear mapping via a GAN based deep learning model. The GAN model might be derived from a model such as described by T. Wang et al (‘High-Resolution Image Synthesis and Semantic Manipulation with Conditional GANs,’ 2018 IEEE/CVF Conference on Computer Vision and Pattern Recognition, 2018, pp. 8798-8807, doi: 10.1109/CVPR.2018.00917). However, in the radiologic image translation domain, known methods are affected by the challenge of generating high-resolution images; and lack the detail and realistic textures of high-resolution results. In their work (ref ‘Comparison of Supervised and Unsupervised Deep Learning Methods for Medical Image Synthesis between Computed Tomography and Magnetic Resonance Images’, BioMed research international, 2020, 5193707, doi: 10.1155/2020/5193707) Y. Li et al proposed cycle-consistent adversarial networks (‘CycleGAN’) to translate between brain CT and MRI images in a low resolution of 256×256. However, high resolution image is normally required for medical diagnosis.

GANs that are trained with unpaired data, for example in semi-supervised learning, have proven particularly susceptible to risks of introducing artifacts or removing relevant information from an image. These GANs are susceptible to these risks because these GANs entail only an indirect check to verify that the synthesized image shows the same content as in the original image. An illustration for example is by A. Keikhosravi el al (‘Non-disruptive collagen characterization in clinical histopathology using cross-modality image synthesis’, Communications Biology 3, 414 (2020), doi: 10.1038/s42003-020-01151-5). This GAN comparison study shows that supervised paired image-to-image translation yields higher image quality in the target domain than the semi-supervised unpaired image-to-image translation.

CycleGAN, trained with unpaired data, is a GAN model capable of translating an image from one domain to another. The use of CycleGAN for image-to-image translation risks mismatch between the distribution of disease in both domains.

Furthermore a CycleGAN generated image is found to lose a certain level of low amplitude and high frequency details that are present in the source image (C. Chu (‘CycleGAN, a Master of Steganography’, NIPS 2017 Workshop)). While this appears a minor information loss visually, it can affect downstream medical image analysis.

The present invention overcomes such problems. It provides manufacture agnostic means to learn a translation mapping between paired for-processing and for-presentation images using GAN. The trained GAN can convert a for-processing image to a vendor-neutral for-presentation image. The present invention further serves as a standardization framework to alleviate differences as well ensuring comparable review across different radiography equipment, acquisition settings and representations.

### SUMMARY OF THE INVENTION

According to a first aspect of the invention there is a system and method for learning a translation mapping between for-processing and for-presentation image pairs via generative adversarial network (GAN) based deep learning system.

According to a second aspect of the invention there is a generative adversarial network (GAN) comprising a first neural network as a generator and a second neural network as a discriminator configured to train one another to learn a translation mapping between sets of paired for-processing and for-presentation images.

A trained generator may convert a for-processing image to a pseudo for-presentation image with manufacturer neutral visualization.

In the translation of for-processing mammograms to for-presentation mammograms, for example, full-field digital mammography (FFDM) systems may produce both ‘for-processing’ (raw) and real ‘for-presentation’ (processed) image formats. The real for-presentation image may be display optimised for radiologists' interpretation. The real for-presentation image may be processed from the for-processing image via a vendor or manufacturer specific algorithm. Consequently, the real for-presentation images may have look distinctive to each of the vendors of imaging machines and systems. Real for-presentation images from one vendor may look different to real for-presentation images of another vendor even though the same tissue of the same patient is the subject of the images.

The images for training may be arranged in a first set of pairs. in the first set paired for-processing images and real for-presentation images may be in the same size (for example height 512×width 512 pixels) and aligned in pixels whereby pixels at a location (x, y) in respective for-processing and real for-presentation images may have different pixel values but they must represent the same tissue.

Each of the for-processing images is a source image. Each of the real for-presentation images is a target image in a sense that a generator aims to produce pseudo for-presentation images very nearly like the real for-presentation images in the first set. A discriminator attempts to gauge how closely the pseudo for-presentation images resemble the real for-presentation images.

To train the discriminator, the generator may be configured to yield a pseudo for-presentation image A′ from a for-processing image A. The discriminator may be configured to yield a first score measuring the discriminator performance in identifying a real for-processing image from a first set of paired for-processing images and real for-presentation images. The discriminator may be configured to yield a second score measuring the discriminator performance in identifying the pseudo for-processing image from a second set of paired for-processing images and pseudo for-presentation images. Preferably the discriminator is configured to backpropagate the first score and the second score to update weights of the discriminator.

To train the generator, the discriminator may be configured to yield a third score measuring general image quality difference from a/the first set of paired for-processing images and real for-presentation images. The discriminator may be configured to yield a fourth score measuring image feature-level distance from a/the first set of paired for-processing images and real for-presentation images and a/the second set of paired for-processing images and pseudo for-presentation images. Preferably the generator is configured to backpropagate the third score and the fourth score to update weights of the generator.

Weights may be parameters within a neural network of the generator and/or discriminator that transforms input data within the network's layers.

Each source image may be pre-processed into a corresponding normalised image. Preferably the GAN comprises a preprocessor to configured to receive and normalise a source image to yield the for-processing image A. The preprocessor may be configured to perform gamma correction on the source image and then normalise. A level of gamma correction may be determined by a ratio of breast projected area in the source image to a preselected value. Above a preselected value of the ratio the level of gamma correction is lower than below the preselected ratio.

The system and method image translation including the GAN comprising the generator and the discriminator may be trained under supervision to attempt to convert each one of the normalised images into a corresponding one of the paired real for-presentation images. The supervision may be by autonomous backpropagation. Each attempt by the generator may produce a pseudo for-presentation image. The attempts may be imperfect and improve iteratively following correction enabled by the discriminator.

Each pair of the images in the second set of pairs may be individually operated upon by the generator. Each normalised image may be converted into one of the pseudo for-presentation images. Thus each pseudo for-presentation image corresponds to a particular source image because each normalised image corresponds to that particular source image.

The discriminator may compare the difference between each pseudo for-presentation image and each real for-presentation image corresponding to a particular source image. The discriminator may return a difference score to the generator for its update. During training, the difference score decrease, and the decrease in the difference score indicates an increased quality of the pseudo for-presentation images. An increase quality of the pseudo for-presentation images may indicate that they more closely resemble the real for-presentation images to which they correspond. The difference score may decrease after each iteration after which the generator is updated. The difference score may decrease after a majority of the iterations.

During inference a forward pass of the generator G may converts an input normalised image, i.e. a for-processing normalised image A, to a pseudo for-presentation image A′.

The training may help the model to learn a nonlinear mapping from the normalised domain to the target domain. The model may include a function ƒ:(norm→target) where norm refers to the normalised images in the second set of pairs, and target refers to the real for-presentation images in first set of pairs. The function may implement the nonlinear mapping from the normalised domain to the target domain. The function may be modified by the training.

The GAN feature matching loss may be derived from the discriminator. The discriminator may extract first multi-scale (f₀ . . . fₙ) features and second multi-scale (f₀^{d} . . . fₙ^{d}) features from a generated pair of a source image and a pseudo for-presentation image. The generated pair may be from the second set. Each layer 0 to ‘n’ may enable extraction of a corresponding first and second multi-scale feature.

The discriminator may also extract another first multi-scale (f₀ . . . fₙ) features and second multi-scale (f₀^{d} . . . fₙ^{d}) features from a real pair. The real pair includes the source image and the real for-presentation image B. The real pair may be from the first set.

The GAN feature matching loss may be the sum of a loss between all paired features, e.g. f₀(A 10, A′ 20), f₀(A 10, B 40), f₀^{d}(A 10, A′ 20), and f₀^{d}(A 10, B 40) etc. The GAN feature matching loss may serve as an additional feedback to the generator G.

For example, paired for-processing images and real for-presentation images may be in the first set of pairs. Included in the first set may be pairs of for-processing images from a particular manufacturer's imaging machine and/or process and/or a particular modality and real for-presentation images from the same manufacturer's imaging machine and/or process and/or a particular modality. The for-processing images may be normalised and then re-paired with the real for-presentation images. After training, the model learns a mapping function from the normalised domain to the real for-presentation image for that particular manufacturer's imaging machine and/or process and/or a particular modality ƒ:(norm→for-presentation image).

Given, for example, for-processing images from a second vendor's imaging machine and/or process and/or particular modality, the same normalisation is applied. During inference, the trained model applies the transform ƒ:(norm→for-presentation image) determined from the first manufacturer's imaging machine and/or process and/or particular modality to convert the normalised for-processing image from the second manufacturer's imaging machine and/or process and/or particular modality to produce pseudo for-presentation images styled like those of the first manufacturer's imaging machine and/or process and/or particular modality.

In the GAN the discriminator may comprise a first path of network layers direct from concatenation of the sets of paired images. The discriminator may comprise a second path of network layer from down-sampled resolution from concatenation of the sets of paired images. The first and second paths may share the same network layers.

The discriminator may be configured to extract first multiscale features for each of the network layers in the first path and/or to extract second multiscale features for each of the network layers in the second path. The discriminator may be configured to utilize the extracted features to compute the first score and the second score in a sum which indicates a capability of the discriminator to distinguish the real for-presentation images from the pseudo for-presentation images. The discriminator is configured to utilize the extracted features to compute the third score and the fourth score in a sum which indicates a capability of the generator to generate pseudo for-presentation images similar to the real for-presentation images.

The system and method for learning a translation mapping between for-processing and for-presentation image pairs generates a pseudo for-presentation image that is highly realistic and indistinguishable from the real for-presentation image. Thus, the GAN model serves as an alternative tool to convert for-processing image for better visualization in the absence of manufacturer software or hardware.

A patient typically has a file of previously acquired for-presentation images. These for-presentation images may have been taken at another facility perhaps with another manufacturer's machine and/or process or by another modality. This file of previously acquired for-presentation images may still be useful in comparison to new pseudo and/or real for-presentation images. The GAN model enables good comparison even if the patient's new images are produced at different facility with another manufacturer's machine and/or process or by another modality.

The pseudo for-presentation image has significantly better contrast than the raw image. So they can be used in training classification or lesion detection models, for example the Breast Imaging Reporting and Data System (BI-RADS) model.

The invention will now be described, by way of example only, with reference to the accompanying figures in which:

### BRIEF DESCRIPTION OF THE FIGURES

FIG. 1A shows a source image of a breast produced by an x-ray machine from a first vendor;

FIG. 1B shows a preprocessed normalised image corresponding the source image in FIG. 1A;

FIG. 1C shows a pseudo for-presentation image derived from the preprocessed normalised image in FIG. 1B;

FIG. 1D shows a real for-presentation image derived from the source image in FIG. 1A by a vendor specific algorithm;

FIG. 2A shows a source image of a breast produced by an x-ray machine from a second vendor;

FIG. 2B shows a preprocessed normalised image corresponding the source image in FIG. 2A;

FIG. 2C shows a pseudo for-presentation image derived from the preprocessed normalised image in FIG. 2B;

FIG. 2D shows a real for-presentation image derived from the source image in FIG. 2A by a vendor specific algorithm;

FIG. 3A shows a source image of a breast produced by an x-ray machine from a third vendor;

FIG. 3B shows a preprocessed normalised image corresponding the source image in FIG. 3A;

FIG. 3C shows a pseudo for-presentation image derived from the preprocessed normalised image in FIG. 3B;

FIG. 3D shows a real for-presentation image derived from the source image in FIG. 3A by a vendor specific algorithm;

FIG. 4A shows a source image;

FIG. 4B shows a gamma converted image of the source image in FIG. 4A;

FIG. 4C shows a normalised image of the gamma converted image in FIG. 4B.

FIG. 5 illustrates a training flow of the GAN based image translation model.

FIG. 6 shows an anatomical view of a discriminator; and

FIG. 7 illustrates a forward pass of the generator.

### DETAILED DESCRIPTION

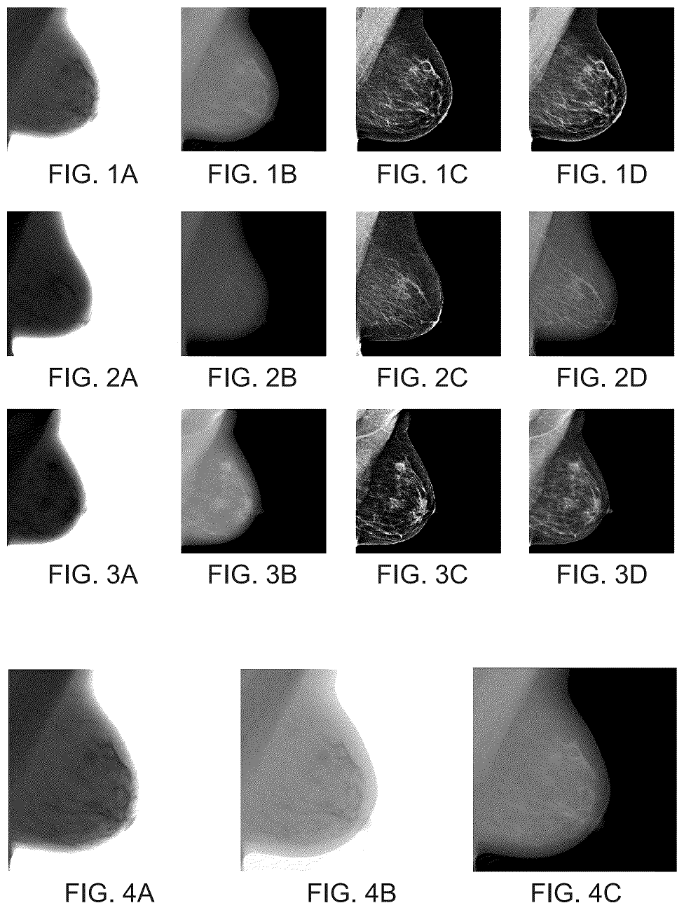

FIG. 1, FIG. 2 and FIG. 3 show a comparison of image visualisation across different vendors: Hologic2DMammo (first row of FIGS. 1A, 1B, 1C, and 1D), SiemensInspiration (second row of FIGS. 2A, 2B, 2C, and 2D) and GEPristina (third row of FIGS. 3A, 3B, 3C, and 3D). Each row of these Figures shows (A) for-processing source image, (B) for-processing normalised image as the input to the generator model in FIG. 5, (C) generated pseudo for-presentation image as the output of the generator and (D) manufacturer specific real for-presentation image. Comparing generated pseudo for-presentation images to corresponding real for-presentation images, the inhomogeneity among the manufacturer specific real for-presentation images is significantly reduced in the GAN generated pseudo for-presentation images.

As seen in FIG. 1, the for-processing images from three vendors are normalised as in FIG. 1B, FIG. 2B, and FIG. 3B The trained model translates the normalised images to the domain of manufacturer and/or modality specific pseudo for-presentation images as in FIG. 1C, FIG. 2C, and FIG. 3C.

As seen in FIG. 1(d), the real for-presentation images from three manufacturers have distinctive visualizations. Comparing FIG. 1C, FIG. 2C, and FIG. 3C to FIG. 1D, FIG. 2D, and FIG. 3D respectively, the normalization step allows a uniform representation of the real for-presentation images from various vendors.

Each one of a plurality of source images of the type shown in FIG. 1A, FIG. 2A, and/or FIG. 3A is normalised. Each one of the normalised images corresponds to the source image from which it was produced. For example the normalised image in FIG. 1B corresponds to the source image in FIG. 1A, the normalised image in FIG. 2B corresponds to the source image in FIG. 2A, and the normalised image in FIG. 3B corresponds to the source image in FIG. 3A. In this way the second set of pairs of images is produced. Each pair in the second set comprises a source image and a corresponding normalised image.

FIG. 1C shows a pseudo for-presentation image which is a result of the generator converting the normalised image shown in FIG. 1B. The generator also converted the normalised images in FIG. 2B and FIG. 3B into the pseudo for-presentation images in FIG. 2C and FIG. 3C respectively.

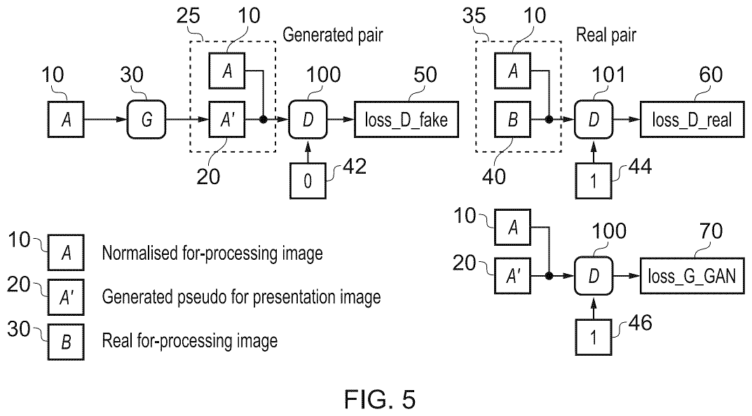

FIG. 4 illustrates image pre-processing for the GAN model. A for-processing image is shown for example in FIG. 4A. The for-processing image is gamma corrected to normalise the contrast between dense and fatty tissue. The gamma corrected imaged is shown for example in FIG. 4B. Then a monochrome conversion is applied resulting in the dense tissue pixel values larger than the fatty tissue pixels. The monochrome conversion is then inverted dark for light resulting in the dense tissue pixel values larger than the fatty tissue pixels. The monochrome conversion is then inverted dark for light so that as shown in FIG. 4C the normalised image is produced.

In an embodiment, and with reference to FIG. 4, an input for-processing mammographic image shown in FIG. 4A is pre-processed to normalise its contrast between the dense (fibroglandular tissue) and fatty tissues, via self-adaptive gamma correction. The resulting normalised for-processing image is shown in FIG. 4B

The GAN comprises a preprocessor to configured to receive and normalise a source image to yield the for-processing image A 10. The preprocessor is configured to perform gamma correction on the source image and then normalise to produce the for-processing image A 10.

Given a source image such as shown in FIG. 4A, a logarithm transform is applied on each pixel as in Eq. (1)

$$I = log ( for - processing image ) \qquad ( 1 )$$

A gamma correction is performed on the logarithm transformed image as in Eq. (2)

$$Gamma Corrected Image = {[ \frac{I - I min}{I max - I min} ]}^{γ} \qquad ( 2 )$$

where values Imin and Imax are the minimum and maximum pixel values respectively in the breast region of the image I.

The GAN is configured to apply a level of gamma correction determined by a ratio of breast projected area in the source image to a preselected value. Above a preselected value of the ratio the level of gamma correction is lower than below the preselected ratio.

For example gamma γ is a self-adaptive variable determined by the breast projected area as in Eq. (3)

$$γ = 0.3 if breast project area \geq 300 {cm}^{2} \qquad ( 3 )$$

$$γ = 0.4 if breast project area < 300 {cm}^{2}$$

A monochrome conversion is applied on the gamma corrected image to obtain the normalised image as in Eq. (4). The normalised image shown for example in FIG. 4B.

$$Normalised Image = 65535 - Gamma Corrected Image \qquad ( 4 )$$

FIG. 4 illustrates the transition from a source for-processing image shown in FIG. 4A to its gamma corrected image shown in FIG. 4B, and finally a normalised image after monochrome conversion to a normalised for-processing image shown in FIG. 4C. Normalised for-processing images are also shown in FIG. 1B, FIG. 2B, and FIG. 3B. The normalised for-processing images have better contrast than the for-processing source images shown in FIG. 1A, FIG. 2A, and FIG. 3A, which benefits the GAN generator to produce high quality pseudo for-presentation images.

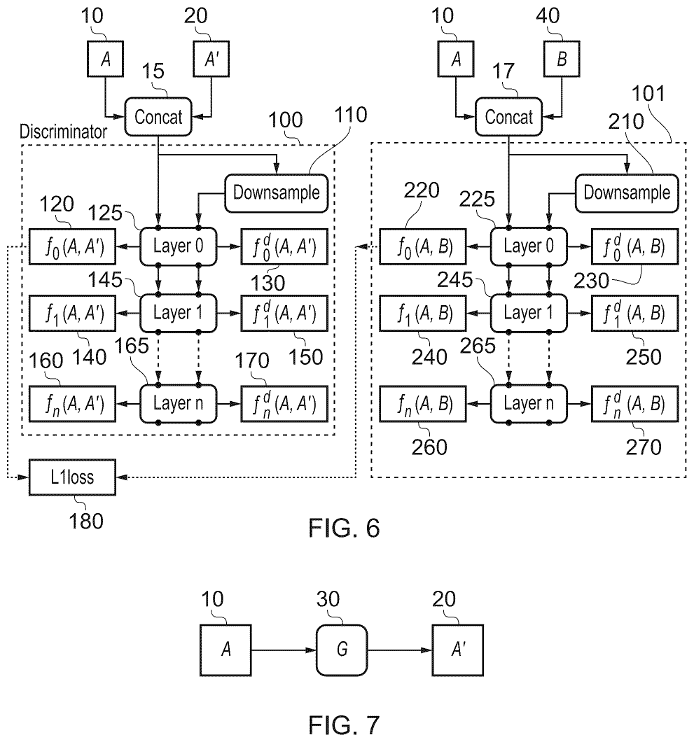

The GAN training flow to implement image translation is abstracted in FIG. 5. Each training instance starts from feeding a normalised for-processing image A 10 into the generator G 30. The generator G 30 is a deep convolutional neural network that contains multiple mathematical operation layers. For example a number ‘n’ operation layers is shown in FIG. 6. The parameters of these operations are randomly initialised and optimised over training. The generator converts a normalised for-processing image to a pseudo for-presentation image A′ 20.

Then, normalised image A 10 and pseudo for presentation image A′ 20 forms a generated pair, which is passed to the discriminator D 100. An anatomical view of discriminator D 100 is shown in FIG. 6. The image pair is evaluated on two paths: a low-level path from the original resolution and a coarse level path from down-sampled resolution. Both paths share the same network layers where each layer computes a feature map (f₀ . . . fₙ 120, 140, 160 from the low-level path and f₀^{d} . . . fₙ^{d} 130, 150, 170 from the coarse level path) encoding the abstracted image information.

Given the generated pair (normalised image A 10, pseudo for-presentation image A′ 20), the discriminator D 100 utilizes the extracted features to compute a probability of its input being fake. The probability is compared with a supervised ground truth label 0 42 shown in FIG. 5. The distance between the probability and the ground truth is denoted as a loss value. This loss value is shown by variable loss_D_fake 50 in FIG. 50.

Similarly, the discriminator D 101 computes loss_D_real 60 when its input is a pair of for processing normalised image A 10, and real for-presentation image B 30. Then, the loss_D_fake 50 and loss_D_real 60 are simply summed together as an overall score to reflect the discriminator's capability in distinguishing real for-presentation images B 30 from generated pseudo for-presentation images A′ 20 respectively. For example the score reflects the discriminator's capability to distinguish pseudo for-presentation image shown in FIG. 1C, FIG. 2C, and FIG. 3C from real for-presentation image shown in FIG. 1D, FIG. 2D, and FIG. 3D respectively. At the initial stage of training, the discriminator D 100, 101 will have a high loss and poor performance. Over training, the loss will decrease, indicating an improved performance.

As discriminator D 100, 101 aims to separate generated pseudo for-presentation images from their real counterparts, the generator G 30 aims to generate realistic for-presentation images A′ 20 to fool the discriminator D100, 101. As shown in FIG. 5 the generator G 30 is updated via a generative adversarial loss loss_G_GAN 70. The generator G 30 may also be updated via a feature matching loss loss_G_Feat. Similar to loss_D_fake 50, loss_G_GAN 70 is computed from the discriminator D 100 with a generated pair (A 10, A′ 20) and a supervised label 1 46, thereby measuring how likely the discriminator identifies the generated image as a real image.

FIG. 5 illustrates a training flow of the GAN based image translation model. The generator G 30 translates a normalised for-processing normalised image A to a pseudo for-presentation image A′ 20. The quality of the pseudo for-presentation image A′ 20 is evaluated by a discriminator D 100 operating on a first input pair and the discriminator D 101 operating on a second input pair.

There is one discriminator. In order to show in FIGS. 5 and 6 where the discriminator 100 is operating on the first input pair (A 10, A′ 20) from when the discriminator 101 is operating on the second input pair (A 10, B 40), the discriminator has two annotation numbers 100 and 101.

The discriminator D 100 with first inputs operates with the first inputs being the for-processing image A 10 and the corresponding pseudo for-presentation image A′ 20. The discriminator D 101 with second inputs operates with the second inputs being the for processing image A 10 and the corresponding real for-presentation image B 40. As shown in FIG. 6 when operating with the first input pair (A 10, A′ 20) and also when operating with the second inputs (A 10, B 40), the discriminator 100, 101 has a number n of layers 125, 145, 165, 225, 245, 265.

It may be seen in FIG. 5 that the performance of the discriminator 100, 101 is driven by its loss in determining real image pair (A 10,B 40) as variable loss_D_real 60 as well the loss in determining the generated pseudo image pair (A, 10 A′ 20) as variable loss_D_fake 60. The generator 30 aims to produce a pseudo for-presentation image A′ 20 to fool the discriminator 100. The performance of the generator 30 in accomplishing this aim is improved by feedbacks from discriminator 100 over training as a generative adversarial loss as loss_G_GAN 70.

FIG. 6 aids illustration of deriving the GAN feature matching loss from discriminators. As shown the discriminator 100 extracts first multi-scale (f₀ . . . fₙ) 120, 140, 160 features and second multi-scale (f₀^{d} . . . fₙ^{d}) 130, 150, 170 features from generated pair (A 10, A′ 20). Each layer 0 to ‘n’ enables extraction of a corresponding first and second multi-scale feature. The discriminator 100 also extracts another first multi-scale (f₀ . . . fₙ) 220, 240, 260 features and second multi-scale (f₀^{d} . . . fₙ^{d}) 230, 250, 270 features from real pair (A 10, B 40) respectively. The generated pair includes the pseudo for-presentation image A′ 20 generated by the generator 30. The GAN feature matching loss is the sum of L1loss 180 between all paired features, e.g. f₀ (A 10, A′ 20) 120, 140, 160 and f₀ (A 10, B 40) 220, 240, 260, f₀^{d}(A 10, A′ 20) 130, 150, 170 and f₀^{d} (A 10, B 40) 230, 250, 270, etc. The GAN feature matching loss serves as additional feedback to the generator G 30.

To further improve the performance of the generator G 30, a feature matching loss loss_G_Feat is also propagated to the generator. The feature matching loss_G_Feat measures the difference of the generated pseudo for-presentation images A′ 20 and real for-presentation images B 30 in abstracted feature levels. These features are extracted from the discriminator 100, 101 as shown in FIG. 6.

A generated pseudo pair produces features ƒ₀(A, A′) . . . ƒₙ(A, A′) 120, 140, 160 from the low-level path and ƒ₀^{d}(A, A′) . . . ƒₙ^{d}(A, A′) 130, 150, 170 from the coarse-level path. They are summed for all levels 0 to ‘n’ as L1loss 180. A real pair produces features ƒ₀(A, B) . . . ƒₙ(A, B) 220, 240, 260 and ƒ₀^{d}(A, B) . . . ƒₙ^{d}(A, B) 230, 250, 270 from the low-level and coarse-level paths respectively. They are also summed for all levels as L1loss 180. The feature loss is defined as Eq. (5) as the sum of features from the generated pseudo pair and real pair.

$$loss\_G \_Feat = {\sum }_{i = 0}^{n} L 1 loss ( {f}_{i} ( A , {A}^{′} ) , {f}_{i} ( A , B ) ) + {\sum }_{i = 0}^{n} L 1 loss ( {f}_{i}^{d} ( A , {A}^{′} ) , {f}_{i}^{d} ( A , B ) ) \qquad ( 5 )$$

Once the GAN model is trained, the generator G 30 is taken for the inference as in FIG. 7. During inference, a forward pass of the generator G 30 converts the input normalised image A 10 to a pseudo for-presentation image A′ 20.

The training flow may be described in the pseudo codes below:

For for-processing normalised image A 10, target for-presentation image B 30 in folder_source_norm, folder_target:
Train discriminator D 100, 101:
   pass A 10 to generator G 30 to yield generated psuedo for-presentation image A′ 20
   pass (A 10, A′ 20) to D 100 to yield a score loss_D_fake 50 (measuring D 100 performance in identifying fake image)
   pass (A 10, B 30) to D 101 to yield a score loss_D_real 60 (measuring D performance in identifying real image)
   backpropagate loss_D_fake 50 and loss_D_real 60 to D to update weights of the discriminator D
Train generator G 30:
   pass (A 10, A′ 20) to D to yield loss_G_GAN 70 (measuring general image quality difference)
   pass (A 10, A′ 20) and (A 10, B 30) to D 101 to yield loss_G_GAN_Feat 180 (measuring image feature-level distance)
   backpropagate loss_G_GAN 70 and loss_G_GAN_Feat 180 to G 30 to update weights of the generator G.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

1. A Generative Adversarial Network (GAN) comprising a first neural network as a generator and a second neural network as a discriminator configured to train one another to learn a translation mapping between sets of paired for-processing and for-presentation images.

2. A GAN according to claim 1 wherein to train the discriminator;
the generator is configured to yield a pseudo for-presentation image A′ from a for-processing image A,
the discriminator is configured to yield a first score measuring the discriminator performance in identifying a real for-processing image from a first set of paired for-processing images and real for-presentation images,
the discriminator is configured to yield a second score measuring the discriminator performance in identifying the pseudo for-processing image from a second set of paired for-processing images and pseudo for-presentation images,
the discriminator is configured to backpropagate the first score and the second score to update weights of the discriminator.

3. A GAN according to claim 1 wherein to train the generator;
the discriminator is configured to yield a third score measuring general image quality difference from the first set of paired for-processing images and real for-presentation images,
the discriminator is configured to yield a fourth score measuring image feature-level distance from the first set of paired for-processing images and real for-presentation images and the second set of paired for-processing images and pseudo for-presentation images,
the generator is configured to backpropagate the third score and the fourth score to update weights of the generator.

4. A GAN according to claim 2 comprising a preprocessor configured to receive and normalise a source image to yield the for-processing image A.

5. A GAN according to claim 4 wherein the preprocessor is configured to perform gamma correction on the source image and then normalise.

6. A GAN according to claim 5 wherein the preprocessor is configured to apply a level of gamma correction determined by a ratio of breast projected area in the source image to a preselected value.

7. A GAN according to claim 6 wherein above a preselected value of the ratio the level of gamma correction is lower than below the preselected ratio.

8. A GAN according to claim 1, wherein the discriminator comprises a first path of network layers direct from concatenation of the sets of paired images.

9. A GAN according to claim 1, wherein the discriminator comprises a second path of network layers from down-sampled resolution from concatenation of the sets of paired images.

10. A GAN according to claim 9, wherein the first and second paths share the same network layers.

11. A GAN according to claim 8, wherein the discriminator is configured to extract first multiscale features for each of the network layers in the first path and/or to extract second multiscale features for each of the network layers in the second path.

12. A GAN according to claim 11, where the discriminator is configured to utilize the extracted features to compute the first score and the second score in a sum which indicates a capability of the discriminator to distinguish the real for-presentation images from the pseudo for-presentation images.

13. A GAN according to claim 11, where the discriminator is configured to utilize the extracted features to compute a third score measuring general image quality difference from the first set of paired for-processing images and real for-presentation images, and a fourth score measuring image feature-level distance from the first set of paired for-processing images and real for-presentation images and the second set of paired for-processing images and pseudo for-presentation images in a sum which indicates a capability of the generator to generate pseudo for-presentation images similar to the real for-presentation images.
